(19) United States
(12) Patent Application Publication
(10) Pub. No.: US 2021/0085587 A1
(43) Pub. Date: Mar. 25, 2021

(54) A TANNING COMPOSITION

(71) Applicant: UBEAUTY GLOBAL, Glasgow (GB)

(72) Inventor: Rebecca MONE, Glasgow (GB)

(73) Assignee: UBEAUTY GLOBAL, Glasgow (GB)

(21) Appl. No.: 16/967,582

(22) PCT Filed: Feb. 7, 2019
(86) PCT No.: PCT/EP2019/053004

§ 371 Date: Aug. 5, 2020

(30) Foreign Application Priority Data
Feb. 7, 2018 (GB) 1801994.3
May 9, 2018 (GB) 1807552.3

(51) Int. Cl. A61K 8/60; A61K 8/73; A61K 8/31; A61K 8/34; A61K 8/35; A61Q 19/04

(52) U.S. Cl. CPC A61K 8/60; A61K 8/733; A61K 8/31; A61K 2800/92; A61K 8/35; A61Q 19/04; A61K 8/347

(57) ABSTRACT

The invention provides a tanning composition for oral administration by humans, the composition being a chewable pastille comprising:

(i) a chewable base comprising a gelling agent and a sugar and/or sugar alcohol; and
(ii) one or more skin colour-promoting agents.

The sugar is typically a mixture of glucose and sucrose.

The gelling agent may be is selected from gelatin, pectin, agar, alginate and carrageenan, and mixtures thereof.

The skin colour-promoting agents may comprise one or more of the carotenoids β-carotene, lycopene, zeaxanthin, lutein and astaxanthin, tyrosine, PABA and copper.

This invention relates to a tanning composition and more particularly to a tanning composition for oral administration by humans, as well as methods of cosmetic tanning using the compositions.

### BACKGROUND OF THE INVENTION

The use of orally ingestible tanning tablets to promote tanning of human skin is well known. Tanning tablets can contain substances that are precursors to, or promote the formation of, melanin in melanocytes in the skin. Alternatively, or additionally, tanning tablets can contain natural pigment substances such as carotenoids that are deposited in the epidermis or sub-cutaneous fat layers and provide the skin with a more highly coloured appearance akin to a tan.

For example, Holland & Barrett “Tan Tablets” contain a blend of copper (in the form of cupric oxide), L-tyrosine and para-aminobenzoic acid (PABA).

“Perfectil Plus Protan”, available from Vitabiotics, UK, contains a mixture of L-tyrosine, lycopene, grape seed extract, copper and biotin.

French patent application FR 2320732 (Tedgui-Zagame) discloses orally ingestible tanning compositions containing carotenoids. While is it stated that the compositions can take various forms, only capsules are given by way of example.

French patent application FR 2339403 (Seguin) describes orally ingestible skin-colouring compositions containing coloured extracts from vegetables such as carrots. The extracts are used to form tablets, dragées or capsules.

French patent application FR 2749757 and International patent application WO 97/47278 (both in the name of Laboratoire Oenobiol) disclose orally ingestible compositions of carotenoids and sun-protectants. The compositions are preferably formulated in soft capsules.

UK Patent application GB 2274235 (Neo-Life Company of America) discloses dietary supplements containing powdered vegetable material in an edible oil base, where the powdered vegetable material contains the carotenoids alpha-carotene, beta carotene and lycopene. The dietary supplements are formulated in gelatin capsules. No mention is made of any tanning effect.

WO 2012/116992 (DSM IP Assets BV) discloses oral compositions containing Steviol or Isosteviol as skin tanning agents. The Steviol and Isosteviol are said to activate melanin formation in melanocytes. The oral compositions can take any form suitable for oral administration, including food products, foodstuffs, and nutritional supplements, and can contain skin tanning agents such as lutein, lycopene, astaxanthin, canthaxanthin, zeaxanthin, beta-carotene, L-tyrosine, vitamin E, vitamin D and copper as well as the Steviol or Isosteviol. The formulations specifically exemplified in WO 2012/116992 are a tablet, a hard gelatine capsule, a soft drink and a fortified non-baked cereal bar. The tablet, capsule and cereal bar contain only Steviol as the skin tanning active ingredient while the soft drink contains Steviol and beta-carotene.

In addition, there are a number of other documents which describe compositions containing skin-colouring compounds.

For example, FR2722094 describes carotenoid-containing compositions for use in prolonging the tanning effect of sun on the skin. In the specific examples described in the application, Examples 1 and 2 are capsules which do not contain a sugar and Example 3 is a powder.

CN102669377 describes a lutein supplement in the form of a ‘soft candy’. The document notes that lutein can be used to protect the retina of the eye from UV damage and therefore can be used to treat/prevent age-related macular degeneration, and is not concerned with providing a composition for changing the colour of the consumer's skin.

CN108112762 describes a “soft candy” containing phosphatidylserine and/or omega-3. The candy also contains sweeteners, gelling/embedding agents. Whilst beta-carotene is mentioned as a component of the jelly sweet, no mention of a role for the jelly sweets in tanning is made.

CN103798565 describes a mung bean jelly powder for production of mung bean jelly. However, this powder does not contain any carotenoid compounds.

US 2005/031557 describes a composition comprising the three carotenoids β-carotene, lutein and lycopene for use in sun protection. Whilst the compositions described in this document may comprise gelatin and sugar, these compositions are tablets rather than chewable pastilles.

### The Invention

The present invention provides an improved tanning composition for providing cosmetic tanning of human skin.

Accordingly, in a first aspect, the invention provides a tanning composition for oral administration by humans, the composition being a chewable pastille comprising:

(i) a chewable base comprising a gelling agent and a sugar and/or sugar alcohol; and

(ii) one or more skin colour-promoting agents.

The term “chewable pastille” as used herein refers to a solid dosage form that has a chewy generally gummy texture and can be chewed without crumbling in the manner that conventional pharmaceutical tablets do when chewed. The properties of the pastilles are thus akin to those of gummi candies. The chewable pastille is formulated so that it dissolves gradually in the mouth as it is chewed. In contrast to known orally ingestible tanning compositions which are intended to be swallowed intact, because the chewable pastille of the invention is held in the mouth rather than being swallowed, at least a proportion of the active ingredients of the pastilles (e.g. the skin colour-promoting agents) can be absorbed through the oral mucosa and in particular the membranes lining the cheeks. The inventor of the present invention has found that administration of certain colour-promoting agents (e.g. β-carotene) in the form of suckable or chewable compositions results in a more rapid skin-colouring effect compared to conventional β-carotene tablets (see Example 3A below).

In addition, in conventional tanning capsules or tablets which are ingested whole, the components within the capsules/tablets are subjected to the harsh acidic conditions found in the stomach which can destroy the components in the composition. By contrast, in the chewable/suckable compositions of the invention, the active ingredients are absorbed into the bloodstream through the oral mucosa, this route of administration avoids the acidic conditions in the stomach.

For the avoidance of doubt, the term “pastille” as used herein is not intended to imply any shape limitation: thus the chewable pastilles of the invention can be any shape.

In this application, the terms “gummi candy”, “gummy candy”, “gummi” or “gummy” may be used as synonyms for “chewable pastille”. The term “gummy base” may be used as a synonym for “chewable base”.

References herein to percentages by weight (w/w) refer to the percentage weight relative to the total weight of the chewable pastille, unless indicated to the contrary.

The term “tanning” as used herein refers to the process of changing the colouration of skin so that it has a more tanned or highly coloured appearance. The tanning process of the invention does not necessarily result in an increased melanin pigmentation of the skin, although the components of the pastille may include substances that promote melanin production.

Accordingly, in some embodiments of the invention, the skin colour-promoting agents are coloured substances which, when ingested, are deposited in the epidermis and/or subcutaneous fatty tissue to create the appearance of tanned skin. Examples of such substances are carotenoids. A combination of two or more coloured substances (with different light absorption profiles) can be used to generate a tanned skin appearance with an optimal colour profile. In particular, the inventor has observed that compositions containing only β-carotene cause the skin to turn orange rather than a healthy tanned colour. Therefore, preferably, other carotenoids are included in the composition which counteract the “oranging effect” of β-carotene.

In alternative embodiments of the invention, the skin colour-promoting agents are substances which facilitate natural UV-induced tanning or which promote or facilitate the production of melanin in the melanocytes in the skin. Examples of such substances include tyrosine (more particularly L-tyrosine which is a bio-precursor to melanin), PABA and copper. In still further embodiments of the invention the skin colour-promoting agents are a mixture of (1) coloured substances which, when ingested, are deposited in the epidermis and/or subcutaneous fatty tissue to create the appearance of tanned skin and (2) substances which promote melanin production.

The terms “subject” and “subjects” as used herein refer to animal subjects, more particularly mammalian subjects and most particularly human subjects.

Carotenoids are tetraterpenoid compounds that can be hydrocarbons (as in the carotenes) or may additionally contain oxygen (as in the xanthophylls).

Examples of carotenes include β-carotene (which has a red-orange colour) and lycopene (which has a deep red colour).

Examples of xanthophylls include zeaxanthin (which has a generally orange colour), lutein (which has a yellow-orange colour), and astaxanthin (which has a reddish-pink colour).

In embodiments of the invention where the skin-colour promoting agents comprise coloured substances, such as carotenoids, the compositions typically include two or more (for example, 3, 4 or 5) carotenoids, for example carotenoids selected from β-carotene, lycopene, zeaxanthin, lutein and astaxanthin. In some embodiments of the invention, the compositions contain all five of β-carotene, lycopene, zeaxanthin, lutein and astaxanthin.

In particular, it has been found that when the compositions of the invention comprise all five of these skin-colour promoting agents, an optimal skin tone is generated owing to the different colour absorbance profiles of each of the components. Accordingly, the compositions of the invention may comprise two or more, for example three or more, preferably four or more skin-colour promoting agents selected from the group consisting of β-carotene, lycopene, zeaxanthin, lutein and astaxanthin.

In another embodiment, the compositions may contain at least one carotene and at least one xanthophyll.

Preferably, the carotenoid is other than canthaxanthin.

As mentioned above, the skin colour-promoting agents may take the form of substances which facilitate natural UV-induced tanning or which promote or facilitate the production of melanin in the melanocytes. Examples of such substances include tyrosine, more particularly L-tyrosine, PABA and copper which can be present in the form of a copper salt, such as copper citrate or copper gluconate. Typically, in embodiments of the invention where the skin colour-promoting agents comprise substances which facilitate natural UV-induced tanning or which promote or facilitate the production of melanin in the melanocytes, the compositions include two or more of these substances. In some embodiments of the invention, the compositions comprise L-tyrosine, PABA and copper citrate.

The tanning compositions of the invention may be substantially free of Stevia extracts, components or compounds such as steviol and isosteviol.

The chewable pastille compositions of the invention are chewable and have a generally gummy texture akin to that of gummi (gummy) candies.

The gummy base of the chewable pastille comprises a gelling agent and a sugar and/or sugar alcohol and typically also an amount of water. The proportions of the gelling agent and the sugar and the amount of water are selected so that, after processing of the components of the composition, the desired gummy texture is achieved.

The sugar can be a monosaccharide such as glucose or fructose, or a disaccharide such as sucrose, or a mixture of monosaccharide(s) and disaccharide(s).

The sugar alcohol, when present, can be for example selected from glycerol, erythritol, threitol, arabitol, xylitol, ribitol, mannitol, sorbitol, galactitol, fucitol, iditol and inositol, a particular sugar alcohol being xylitol. The sugar alcohol can be used in combination with one or more sugars. For example, in order to reduce the propensity of the compositions to promote tooth decay, at least part of the sugar can be replaced by a sugar alcohol such as xylitol, provided that the ability of the compositions to form a chewable pastille of gummy consistency is not compromised.

In one embodiment, the chewable (gummy) base comprises a gelling agent and a mixture of sucrose and glucose.

The sugar and/or sugar alcohol typically constitute from about 60% (w/w) to about 95% (w/w) of the total weight of the pastille composition.

More usually, the sugar and/or sugar alcohol constitute from about 70% (w/w) to about 90% (w/w) of the composition.

More particularly, the sugar and/or sugar alcohol may constitute from about 75% (w/w) to about 85% (w/w) of the composition.

In one particular embodiment, the sugar and/or sugar alcohol constitute from about 78% (w/w) to about 82% (w/w) of the composition.

In each of the foregoing embodiments, the chewy base may contain no sugar alcohols, or it may contain a minor proportion of sugar alcohols.

In one particular embodiment, the chewy base contains one or more sugars but no sugar alcohols.

In another particular embodiment, the chewable base comprises a gelling agent and a mixture of sucrose and glucose wherein the mixture of sucrose and glucose constitutes from about 75% (w/w) to about 85% (w/w) of the composition, and more particularly from about 78% (w/w) to about 88% (w/w) of the composition.

When the sugar is a mixture of monosaccharide and disaccharide (e.g. glucose and sucrose), the weight ratio of the monosaccharide:disaccharide (e.g. glucose:sucrose) may be in the range from 4:1 to 1:4, more usually from 3:1 to 1:3, for example from 2:1 to 1:2, and more particularly, from 1.5:1 to 1:1.5.

In one embodiment, the chewable base comprises a gelling agent and a mixture of sucrose and glucose wherein the glucose is present in an amount of 32-40% (w/w) of the total weight of the composition and the sucrose is present in an amount of 40-48% (w/w) of the total weight of the composition.

The chewable base typically contains water in an amount of up to about 15% (w/w) of the total weight of the composition. More usually, the amount of water is in the range from about 4% (w/w) to about 12% (w/w), and more particularly, from about 5% (w/w) to about 10% (w/w).

The gelling agent is typically a food-grade gelling agent and is usually obtainable from natural sources.

For example, the gelling agent may be selected from gelatin, pectin, agar, alginate and carrageenan, and mixtures thereof.

In one embodiment, the gelling agent is gelatin.

In other embodiments, the gelling agent is selected from pectin, agar, alginate or carrageenan, and a mixture of one or more thereof.

In one particular embodiment, the gelling agent is pectin.

The gelling agent is typically present in an amount of about 2% (w/w) to about 6% (w/w) of the total weight of the composition. More particularly, the gelling agent may be present in an amount of about 2.5% (w/w) to about 5% (w/w), for example from 3-4% (w/w).

Citric acid may be included in order to assist the gelling agent (particularly pectin) to exert its gelling effect on the sugar. The amount of citric acid may be from 0.1% (w/w) to 5% (w/w), more typically from 0.5% (w/w) to about 3% (w/w), and preferably from 1% to 3% (w/w).

In embodiments of the invention wherein the skin colour-promoting agents comprise coloured substances skin colour-promoting agents are typically present in the compositions in a total amount of from about 0.2% (w/w) to about 2% (w/w) of the total weight of the composition. More usually, the skin colour-promoting agents are present in the compositions in a total amount of from about 0.4% (w/w) to about 1.5% (w/w), for example in an amount from about 0.5% (w/w) to about 1% (w/w).

In embodiments of the invention wherein the skin colour-promoting agents comprise coloured substances, the skin colour-promoting agents may comprise:

(a) β-carotene in an amount of 0.1%-0.8% (w/w);

(b) lutein in an amount of 0%-0.15% (w/w);

(c) lycopene in an amount of 0%-0.1% (w/w);

(d) astaxanthin in an amount of 0%-0.1% (w/w);

(e) zeanxhantin in an amount of 0%-0.1% (w/w); and

(f) a physiologically acceptable copper salt in an amount of 0%-0.05% (w/w);

provided that the total amount of skin colour-promoting agent is the range from 0.5%-1% (w/w).

In one embodiment, the skin colour-promoting agents are selected from:

(a) β-carotene in an amount of 0.1%-0.8% (w/w);

(b) lutein in an amount of 0.02%-0.15% (w/w);

(c) lycopene in an amount of 0.02%-0.1% (w/w);

(d) astaxanthin in an amount of 0.01%-0.1% (w/w);

(e) zeanxhantin in an amount of 0.002%-0.1% (w/w); and optionally

(f) a physiologically acceptable copper salt in an amount of 0.005%-0.05% (w/w);

provided that the total amount of skin colour-promoting agent is the range from 0.5%-1% (w/w).

In another embodiment, the skin colour-promoting agents are selected from:

(a) β-carotene in an amount of 0.3%-0.8% (w/w);

(b) lutein in an amount of 0.05%-0.15% (w/w);

(c) lycopene in an amount of 0.03%-0.1% (w/w);

(d) astaxanthin in an amount of 0.01%-0.1% (w/w);

(e) zeanxhantin in an amount of 0.005%-0.1% (w/w); and optionally

(f) a physiologically acceptable copper salt in an amount of 0.01%-0.03% (w/w)

provided that the total amount of skin colour-promoting agent is the range from 0.5%-0.9% (w/w).

In other embodiments of the invention, the tanning compositions comprise:

(a) β-carotene in an amount of 2.5 mg-20 mg (w/w);

(b) lutein in an amount of 0 mg-3.75 mg (w/w);

(c) lycopene in an amount of 0 mg-2.5 mg (w/w);

(d) astaxanthin in an amount of 0 mg-2.5 mg (w/w);

(e) zeanxhantin in an amount of 0 mg-2.5 mg (w/w); and

(f) a physiologically acceptable copper salt in an amount of 0 mg-1.25 mg (w/w);

provided that the total amount of skin colour-promoting agent is in the range from 12.5 mg-25 mg (w/w).

In addition to the skin colour-promoting agents defined above, the pastille compositions may also comprise any one or more biologically active substances selected from vitamins, minerals and substances that promote natural tanning by assisting in the production of melanin.

As mentioned above, examples of substances that promote natural tanning include tyrosine and PABA.

The amino acid tyrosine (e.g. L-tyrosine) can be present in an amount of 0.5% to 15% (w/w), typically 0.5% to 10% (w/w), for example 0.5 to 5% (w/w) of the pastille composition, and more usually from 1% to 4% (w/w) of the pastille composition.

The compound 4-aminobenzoid acid (PABA) can be present in an amount of 0.5 to 5% (w/w) of the pastille composition, for example from 1% to 4% (w/w).

In embodiments of the invention wherein the skin colour-promoting agents comprise substances which facilitate natural UV-induced tanning or which promote or facilitate the production of melanin in the melanocytes in the skin, the skin colour-promoting agents are typically present in the compositions in a total amount of from about 1% (w/w) to about 10% (w/w) of the total weight of the composition. More usually, the skin colour-promoting agents are present in the compositions in a total amount of from about 1% (w/w) to about 8% (w/w), for example in an amount from about 1% (w/w) to about 7% (w/w).

In embodiments of the invention wherein the skin colour-promoting agents comprise substances which facilitate natural UV-induced tanning or which promote or facilitate the production of melanin in the melanocytes in the skin, the skin colour-promoting agents may comprise one or more of:

(a) L-Tyrosine in an amount of 0.5%-15% (w/w);

(b) PABA in an amount of 0.5%-5% (w/w); and

(c) Copper Citrate in an amount of 10%-20% (w/w).

In embodiments of the invention wherein the skin colour-promoting agents comprise substances which facilitate natural UV-induced tanning or which promote or facilitate the production of melanin in the melanocytes in the skin, the skin colour-promoting agents may comprise one or more of:

(a) L-Tyrosine in an amount of 1%-15% (w/w);

(b) PABA in an amount of 0.5%-5% (w/w); and

(c) Copper Citrate in an amount of 0.01%-0.02% (w/w).

In another embodiment, the skin colour-promoting agents may comprise one or more of:

(a) L-Tyrosine in an amount of 2%-12% (w/w);

(b) PABA in an amount of 1%-3% (w/w); and

(c) Copper Citrate in an amount of 0.012%-0.018% (w/w).

In a further embodiment, the skin colour-promoting agents may comprise one or more of:

(a) L-Tyrosine in an amount of 10% (w/w);

(b) PABA in an amount of 2% (w/w); and

(c) Copper Citrate in an amount of 0.015% (w/w).

In embodiments of the invention wherein the skin colour-promoting agents comprise substances which facilitate natural UV-induced tanning or which promote or facilitate the production of melanin in the melanocytes in the skin, the skin colour-promoting agents may comprise one or more of:

(a) L-Tyrosine in an amount of 1%-8% (w/w);

(b) PABA in an amount of 0.5%-5% (w/w); and

(c) Copper Citrate in an amount of 10%-20% (w/w).

In another embodiment, the skin colour-promoting agents may comprise one or more of:

(a) L-Tyrosine in an amount of 2%-6% (w/w);

(b) PABA in an amount of 1%-3% (w/w); and

(c) Copper Citrate in an amount of 12%-18% (w/w).

In a further embodiment, the skin colour-promoting agents may comprise one or more of:

(a) L-Tyrosine in an amount of 4% (w/w);

(b) PABA in an amount of 2% (w/w); and

(c) Copper Citrate in an amount of 15% (w/w).

In other embodiments of the invention, the skin colour-promoting agents may comprise one or more of:

(a) L-Tyrosine in an amount of 25 mg-200 mg (w/w);

(b) PABA in an amount of 12.5 mg-125 mg (w/w); and

(c) Copper Citrate in an amount of 0.25 mg-0.50 mg (w/w).

In the foregoing three embodiments, preferably the compositions contain at least two of components (a)-(c), more preferably all three of components (a)-(c).

In addition to the skin colour-promoting agents defined above, the pastille compositions may also comprise any one or more biologically active substances selected from vitamins and minerals.

Examples of vitamins that may be included in any of the compositions of the present invention are vitamin B2 (riboflavin), vitamin B6, vitamin C and vitamin E. A further example is vitamin D. In addition, vitamin B2 (riboflavin) forms a yellow solution when dissolved in water. Therefore, the addition of vitamin B2 to the compositions of the invention not only provides the consumer with a source for this vitamin (to help them reach the recommended daily intake), but also complements the other skin colour-promoting agents within the composition to result in a desirable colour of the consumer's skin.

Examples of minerals (in addition to copper) that can be included are zinc and selenium.

Vitamin B6 can be present in an amount of 0.01% to 0.05% (w/w) of the total weight of the pastille composition, and more usually in an amount from 0.02% to 0.04% (w/w).

Vitamin B2 can be present in an amount of 0.01% to 0.05% (w/w) of the total weight of the pastille composition, and more usually in an amount from 0.02% to 0.04% (w/w).

Vitamin B12 can be present in an amount of 0%-0.05% (w/w) of the total weight of the pastille composition, and more usually in an amount from 0.00002% to 0.0004%.

In some embodiments, vitamin C can be present in an amount of 1% to 3% (w/w) of the total weight of the pastille composition, and more usually from 1.5% to 2% (w/w).

In other embodiments, vitamin C can be present in an amount of 0.1% to 3% (w/w) of the total weight of the pastille composition, and more usually from 0.2 to 2% (w/w).

Vitamin D can be present in an amount of 0-0.005% (w/w) of the total weight of the pastille composition, and more usually in an amount from 0.0001-0.0005% (w/w).

Vitamin E can be present in an amount of 0.1% to 5% (w/w) of the total weight of the pastille composition, and more usually from 0.2% to 0.4%.

Zinc can be present in an amount of 0.02% to 0.08% (w/w) of the total weight of the pastille composition, and more usually from 0.04% to 0.07% (w/w).

Selenium can be present in an amount from 0.0005% (w/w) to 0.002% (w/w) of the total weight of the composition, and more usually from 0.008% to 0.0015% (w/w).

In compositions of the invention containing coloured substances as the skin colour-promoting agents as well as L-tyrosine, the conversion of L-tyrosine into melanin may be assisted by the presence of vitamin C, PABA and copper. In a preferred embodiment, the pastille composition comprises one or more (for example two or more, or three or more) skin colour-promoting agents selected from:

(a) β-carotene;

(b) lutein;

(c) lycopene;

(d) astaxanthin; and

(e) zeanxhantin

together with:

(f) copper;

(g) L-tyrosine;

(h) vitamin C; and optionally

(i) 4-aminobenzoic acid.

In the foregoing embodiment, preferably the compositions contain at least two of components (a)-(e), more preferably at least three of components (a)-(e), still more preferably at least four of components (a)-(e), and most preferably all five of components (a)-(e).

The amounts of each component may be as defined above.

Other substances that can usefully be included in the pastille compositions of the invention include:
flavouring agents—natural or synthetic (e.g. in amounts of 0.5% to 3% (w/w));
colouring agents (e.g. in amounts of 0.01% to 2% (w/w));
vegetable oils such as sunflower oil (e.g. in amounts of 0.002% to 0.01% (w/w)); and
natural waxes such as carnauba wax. (e.g. in amounts of 0.002% to 0.01% (w/w)).

In one particular embodiment, the chewable pastilles have the following composition:

| | % (w/w) of total weight |
|---|---|
| Component | of compositions |
| Sucrose | 35-55 |
| Glucose | 30-50 |
| Water | 5-10 |
| Pectin | 2-5 |
| Flavouring agent | 0-2 |
| Citric acid | 1-3 |
| Colouring agent | 0-0.2 |
| Vegetable (e.g. | 0-0.01 |
| sunflower) oil | |
| Natural wax (e.g. | 0-0.01 |
| Carnauba wax) | |
| Tyrosine | 1-4 |
| Vitamin C | 0.5-4 |
| Vitamin A | 0.2-1 |
| (β-carotene) | |
| Vitamin E | 0.05-0.5 |
| Lutein | 0.05-0.2 |
| Zinc | 0-0.1 |
| Lycopene | 0.02-0.1 |
| Vitamin B2 | 0-0.06 |
| (Riboflavin) | |
| Asthaxin | 0.01-0.05 |
| Copper citrate | 0.005-0.03 |
| Zeanxhantin | 0.005-0.02 |
| Selenium | 0-0.003 |
| Total | 100% |

In another particular embodiment, the chewable pastilles have the following composition:

| | % (w/w) of total weight |
|---|---|
| Component | of compositions |
| Sucrose | 35-55 |
| Glucose | 30-50 |
| Water | 5-10 |
| Tyrosine | 1-5 |
| Pectin | 2-5 |
| PABA | 1-5 |
| Flavouring agent | 0-2 |
| Citric acid | 1-3 |
| Vitamin C | 0.2-2 |
| Vitamin E | 0.05-0.5 |
| Zinc citrate | 0-0.1 |
| Vitamin B2 | 0-0.1 |
| (Riboflavin) | |
| Vitamin B12 | 0-0.05 |
| Copper citrate | 0.005-0.030 |
| Vitamin D | 0-0.005 |
| Vegetable (e.g. | 0-0.01 |
| sunflower) oil | |
| Natural wax (e.g. | 0-0.01 |
| Carnauba wax) | |
| Total | 100% |

The pastilles of the invention may each have a weight of, for example, from 1 grammes to 5 grammes, more usually from 1.5 to 4 grammes. In one embodiment, the pastilles each have a weight of approximately 2.5 grammes.

The pastilles can be prepared by mixing the various components and heating the mixture to form a thick syrupy mass. The heating may be carried out over a period of several hours at a temperature of about 90-120° C. The viscous mass is then poured into moulds and allowed to solidify to form pastilles.

As an alternative, the components of the chewable base (the gelling agent, sugar and/or sugar alcohol and any gelation aids such as citric acid), and optionally some of the less temperature sensitive components can be mixed with water, heated to a temperature of about 90-120° C. and maintained at that temperature until a thick uniform syrupy mass results. The temperature can then be reduced and the remaining components mixed into the syrupy mass. The mass is then poured into moulds and allowed to solidify to form pastilles. By allowing the syrupy mass to cool to some extent before adding the more temperature sensitive components such as carotenoids, oxidation and degradation of such components is minimised or avoided.

The pastille compositions of the invention are administered orally and are held in the mouth whilst sucking or chewing. As the pastilles gradually dissolve, it is believed that at least some of the skin colour-promoting agents are absorbed through the buccal mucosa and therefore enter the blood stream without needing to pass through the stomach and small intestine.

In embodiments of the invention wherein the skin colour-promoting agents comprise coloured substances a typical dosing regime is as follows: an initial dose of 2-3 pastilles, each of 2-3 grammes in weight and having the compositions described above, are consumed for a period of up to a week or more, or for up to two, three or four weeks, or until a desired extent of skin colouring is observed.

The daily dose is then reduced, for example to 1 to 2 pastilles per day, and continued for as long as the consumer wishes to maintain the skin colouration.

In embodiments of the invention wherein the skin colour-promoting agents comprise only substances which facilitate natural UV-induced tanning or which promote or facilitate the production of melanin in the melanocytes in the skin, a typical dosing regime is as follows: an initial dose of 1 or more pastilles, preferably 2 or more, more preferably 3 or more e.g. 3-4, each of 2-3 grammes in weight are consumed each day for a period of 1 day or more, more preferably 3 days or more e.g. 3-4 days, prior to prolonged UV exposure. A further dose of 1 or more pastilles, preferably 2 or more, more preferably 3 or more e.g. 3-4 are consumed for a period of 1 day or more, more preferably 3 days or more e.g. 3-4 days during prolonged UV exposure. Once prolonged UV exposure is terminated a further dose of 1 or more pastilles, preferably 2 or more, more preferably 3 or more e.g. 3-4 may be consumed. At that point the daily dose can either be stopped or reduced, for example to 1 or more pastilles per day.

In another aspect, the invention provides a tanning composition for oral administration by humans, the composition being a gummy candy containing one or more skin colour-promoting agents.

In another aspect, the invention provides a tanning composition for oral administration by humans, the composition being a gummy candy comprising:

(i) a chewable gummy base comprising a gelling agent and a sugar and/or sugar alcohol; and

(ii) one or more skin colour-promoting agents.

In another aspect, the invention provides a method of achieving a desired skin colouration, which method comprises administering to a subject, over a period of at least a week, on a daily basis, a number of chewable pastilles as defined herein sufficient to achieve the desired skin colouration.

In another aspect, the invention provides a method of cosmetic tanning, which method comprises the oral administration to a subject of an effective number of a chewable pastille composition as defined herein.

In another aspect, the invention provides a method of cosmetic skin colour enhancement, which method comprises the oral administration to a subject of an effective number of chewable pastille compositions as defined herein.

In a still further aspect, the invention provides a pastille composition as defined herein for use in a method of medical tanning or skin colour enhancement, which method comprises administering to a subject a number of pastille compositions as defined herein sufficient to bring about a desired extent of tanning or skin colour enhancement.

Examples of methods of medical tanning include the treatment of skin conditions such as vitiligo where natural skin pigmentation has been lost. In patients suffering from vitiligo, the tanning compositions of the invention can assist in masking areas of affected skin, or at least reducing the contrast between de-pigmented and normal skin.

In each of the foregoing methods, the method typically involves a programme of administration comprising a tanning (or skin colour-changing) phase in which a subject ingests a defined number of the chewable pastilles at regular intervals until a desired degree of tanning (or skin colour change) is achieved, followed by a maintenance phase in which a reduced number of chewable pastilles is consumed over a period in which it is desired to maintain the tanning skin colour change.

The tanning (or skin-colouring) phase may last for a week or more, for example, 7, 8, 9, 10, 11, 12, 13 or 14 days, or for 1, 2, 3 or 4 weeks. During this phase, the chewable pastilles may be consumed on a daily basis (for example once daily or twice daily). The number of chewable pastilles consumed per day may be, for example, from 2 to 5, the weights of the pastilles and the amounts of skin colour-promoting agents contained within them being as defined above and in the examples below.

The maintenance phase can take place over any desired period, depending on the period over which a subject wishes to benefit from the tanning or skin colour changing effect. During the maintenance phase, the subject will typically consume only 1 or 2 chewable pastilles daily or at a lower frequency depending on need.

An advantage of the present invention is that it allows subjects to develop a desired skin colour without the need for excessive exposure to the damaging effect of sunlight and ultraviolet radiation. In preferred embodiments of the invention, the chewable pastilles of the invention contain substances that are known to be beneficial to human health. Thus, the ingestion of the pastilles may make a positive contribution to health as well as providing a tanning effect. Furthermore, the formulation of the chewable pastilles enables the active ingredients to be absorbed more readily through the buccal mucosa with the consequence that they should reach the target location (which in some embodiments of the invention is the epidermis and sub-cutaneous layers) quicker and more efficiently than is possible with known tablet and capsule formulations of tanning compounds.

The invention will now be illustrated, but not limited, by reference to the specific Examples below.

### BRIEF DESCRIPTION OF THE DRAWINGS

FIGS. 1A, 2A and 3A are photographs (originally filed in colour) of volunteers' arms at the start of the trial described in Example 3 below.

FIGS. 1B, 2B and 3B are photographs (originally filed in colour) of volunteers' arms at the end of the trial described in Example 3 below.

### EXAMPLES

### Example 1

### Example 1A

A tanning composition was prepared with the following composition.

| | % (w/w) of total weight | Weight (mg) per |
|---|---|---|
| Component | of compositions | two pastilles |
| Sucrose | 44 | 2200 |
| Glucose | 35.6 | 1780 |
| Water | 8.22 | 411 |
| Pectin | 3.6 | 180 |
| Flavouring agent | 1.7 | 85 |
| Citric acid | 1.5 | 75 |
| Artificial | 0.1 | 5 |
| colouring agent | | |
| E124A | | |
| Sunflower oil | 0.005 | 0.25 |
| Carnauba wax | 0.005 | 0.25 |
| Tyrosine | 2.3 | 115 |
| Vitamin C | 1.84 | 92 |
| Vitamin A | 0.576 | 28.8 |
| (β-carotene) | | |
| Vitamin E | 0.276 | 13.8 |
| Lutein | 0.092 | 4.6 |
| Zinc citrate | 0.0576 | 2.88 |
| Lycopene | 0.046 | 2.3 |
| Vitamin B2 | 0.0322 | 1.61 |
| (Riboflavin) | | |
| Astaxanthin | 0.023 | 1.15 |
| Copper citrate | 0.0172 | 0.86 |
| Zeaxanthin | 0.008 | 0.4 |
| Selenium | 0.0012 | 0.06 |
| Totals | 100 | 5000 |

The pastilles were prepared by mixing the various components and heating the mixture to form a thick syrupy mass. which was then poured into moulds and allowed to solidify to form pastilles.

The resulting pastilles have a soft chewy gummy consistency.

### Example 1B

| | % (w/w) of total weight | Weight (mg) per |
|---|---|---|
| Component | of compositions | two pastilles |
| Sucrose | 44 | 2200 |
| Glucose | 35.6 | 1780 |
| Water | 8.9 | 445 |
| Pectin | 3.6 | 180 |
| Flavouring agent | 1.7 | 85 |
| Citric acid | 1.5 | 75 |
| Artificial | 0.01 | 5 |
| colouring agent | | |
| E124A | | |
| Sunflower oil | 0.005 | 0.25 |
| Carnauba wax | 0.005 | 0.25 |
| Tyrosine | 2.0 | 100 |
| Vitamin C | 1.6 | 80 |
| Vitamin A | 0.50 | 25 |
| (β-carotene) | | |
| Vitamin E | 0.24 | 12 |
| Lutein | 0.08 | 4 |
| Lycopene | 0.04 | 2 |
| Vitamin B2 | 0.028 | 1.4 |
| (Riboflavin) | | |
| Astaxanthin | 0.020 | 1 |
| Copper citrate | 0.015 | 0.75 |
| Zeaxanthin | 0.007 | 0.35 |
| Zinc citrate | 0.05 | 2.5 |
| Selenium | 0.0011 | 0.055 |
| Totals | 100 | 5000 |

In a typical dosing regime, 2-3 chewable pastilles (according to Example 1A or 1B) are consumed per day for an initial period of about 10 days, following which an increase in skin colouration is observed as a result of deposition of the zeanxanthin, asthaxin, lycopene and vitamin A in the epidermis and subcutaneous fatty tissue. Once a desired skin colouration has been obtained, a maintenance dose of 1-2 pastilles per day is administered over a period for which a more tanned appearance is required.

### Example 2

### Example 2A

A tanning composition was prepared with the following composition.

| | % (w/w) of total weight | Weight (mg) per |
|---|---|---|
| Component | of compositions | two pastilles |
| Sucrose | 43.23 | 2161.44 |
| Glucose | 35 | 1750 |
| Water | 8.22 | 411 |
| Pectin | 3.6 | 180 |
| Flavouring agent | 1.7 | 85 |
| Citric acid | 1.5 | 75 |
| Sunflower oil | 0.005 | 0.25 |
| Carnauba wax | 0.005 | 0.25 |
| Tyrosine | 4 | 200 |
| Vitamin C | 0.4 | 20 |
| Vitamin E | 0.21 | 10.5 |
| Zinc citrate | 0.06 | 3 |
| Vitamin B2 | 0.056 | 2.8 |
| (Riboflavin) | | |
| Vitamin B12 | 0.000036 | 0.0018 |
| Copper citrate | 0.015 | 0.75 |
| Vitamin D | 0.0002 | 0.01 |
| PABA | 2 | 100 |
| Totals | 100 | 5000 |

The pastilles were prepared by mixing the various components and heating the mixture to form a thick syrupy mass. which was then poured into moulds and allowed to solidify to form pastilles.

The resulting pastilles have a soft chewy gummy consistency.

### Example 2B

| | % (w/w) of total weight | Weight (mg) per |
|---|---|---|
| Component | of compositions | two pastilles |
| Sucrose | 40.2 | 2010 |
| Glucose | 32.0 | 1600 |
| Water | 8.2 | 412 |
| Pectin | 3.6 | 180 |
| Flavouring agent | 1.7 | 85 |
| Citric acid | 1.5 | 75 |
| Sunflower oil | 0.005 | 0.25 |
| Carnauba wax | 0.005 | 0.25 |
| Tyrosine | 10 | 500 |
| Vitamin C | 0.4 | 20 |
| Vitamin E | 0.21 | 10.5 |
| Zinc citrate | 0.06 | 3 |
| Vitamin B2 | 0.05 | 2.8 |
| (Riboflavin) | | |
| Vitamin B12 | 3.6E−05 | 0.0018 |
| Copper citrate | 0.015 | 0.75 |
| Vitamin D | 0.0002 | 0.01 |
| PABA | 2 | 100 |
| Totals | 100 | 5000 |

The pastilles were prepared by mixing the various components and heating the mixture to form a thick syrupy mass. which was then poured into moulds and allowed to solidify to form pastilles.

The resulting pastilles have a soft chewy gummy consistency.

In a typical dosing regime, 3-4 chewable pastilles (according to Example 2A or Example 2B) are consumed per day for 3-4 days prior to exposure, during UV exposure, and optionally after UV exposure (e.g. for up to 3-4 days after UV exposure), following which an increase in skin colouration is observed as a result of an increase in melanin production in the melanocytes in the skin.

### Example 3

### Example 3A

Five volunteers took three pastilles per day for a period of 3 weeks. The pastilles had the composition described in Example 1B with the amounts of the following ingredients amended as detailed below:

| | Weight (mg) per |
|---|---|
| Component | two pastilles |
| Beta-carotene | 30 |
| Lycopene | 1 |
| Lutein | 2 |
| Zeaxanthin | 1 |
| Astaxanthin | 1 |

The weights of all other components not listed in the table above were the same as the amounts used in Example 1B.

Following the end of the trial, 3 of out 5 of the volunteers were dissatisfied with the result due to the orange colour of their palms.

Known tanning tablets containing the same amount of β-carotene do not result in volunteers' palms turning orange and hence it was concluded that the pastilles of the invention show increased rates of absorption of beta-carotene compared to known tablet compositions.

### Example 3B

Ten volunteers took three pastilles per day for a period of 3 weeks. The pastilles had the composition described in Example 1B. The volunteers were not in environments with high sun levels (i.e. they were in environments when they would not normally develop a tanning of the skin) and did not use any other tanning products or equipment (e.g. sun beds).

Following the end of the trial 8 out of 10 of the volunteers were satisfied that their skin had changed colour and 9 out of 10 of the volunteers were satisfied with the resultant colour (i.e. their skin had developed a healthy brown glow rather than an orange colour).

In addition, photographs of the volunteers' skin were taken at the start and end of the trial (see FIGS. 2A and 2B, wherein FIG. 2A was taken before the start of the trial and FIG. 2B was taken at the end of the trial). The tanning effect of the compositions can be seen from a comparison of the photos.

### Example 3C

Twenty volunteers took three pastilles per day for a period of 3 weeks. The pastilles had the composition described in Example 2B. These volunteers took the pastilles in combination with high exposure to natural sunlight or use of sun beds.

Following the end of the trial 17 out of 20 of the volunteers were satisfied that their skin had changed colour to a greater extent than they would have expected by sun exposure or use of sun beds alone).

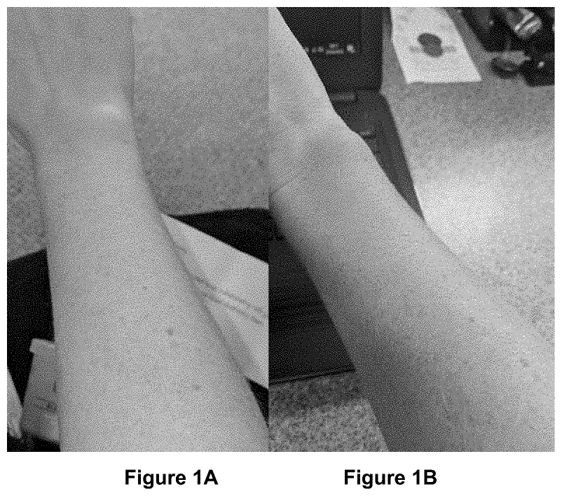

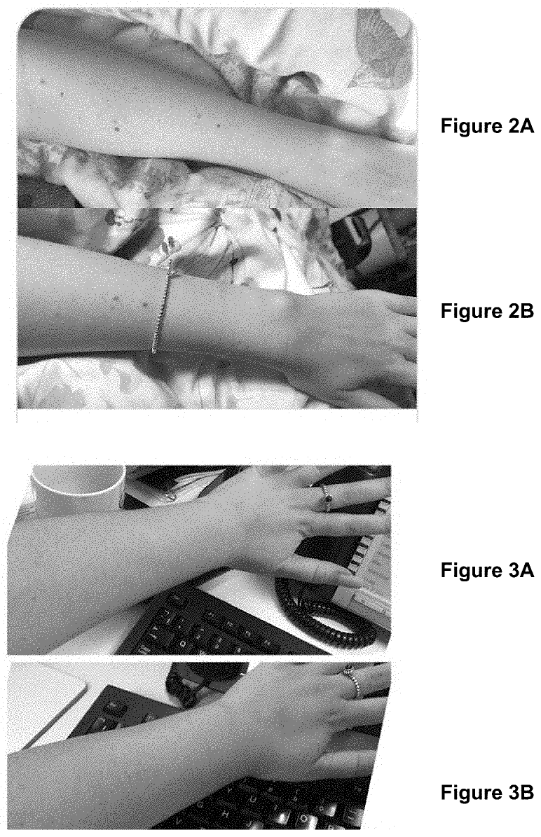

In addition, photographs of the volunteers' skin were taken at the start and end of the trial (see FIGS. 1 and 3, wherein FIGS. 1A and 3A were taken before the start of the trial and FIGS. 1B and 3B were taken at the end of the trial).

### EQUIVALENTS

It will readily be apparent that numerous modifications and alterations may be made to the specific embodiments of the invention described above without departing from the principles underlying the invention. All such modifications and alterations are intended to be embraced by this application.

1. A tanning composition for oral administration by humans, the composition being a chewable pastille comprising:
(i) a chewable base comprising a gelling agent and a sugar and/or sugar alcohol; and
(ii) two or more skin colour-promoting agents selected from β-carotene, lycopene, zeaxanthin, lutein and astaxanthin.

2. A tanning composition according to claim 1 further comprising one or more skin colour-promoting agents comprise one or more of tyrosine, PABA and copper.

3. A tanning composition according to claim 2, wherein the tyrosine is L-tyrosine.

4. A tanning composition according to claim 2, wherein the copper is copper citrate.

5. A tanning composition according to claim 2, wherein the skin colour-promoting agents comprise all of tyrosine, PABA and copper.

6. A tanning composition according to claim 1 further comprising one or more additional carotenoids.

7. A tanning composition according to claim 6 wherein the one or more skin colour-promoting agents comprise three or more carotenoids selected from β-carotene, lycopene, zeaxanthin, lutein and astaxanthin.

8. A tanning composition according to claim 7 containing at least four carotenoids selected from β-carotene, lycopene, zeaxanthin, lutein and astaxanthin.

9. A tanning composition according to claim 8 containing all five carotenoids β-carotene, lycopene, zeaxanthin, lutein and astaxanthin.

10. A tanning composition according to claim 1 wherein the gelling agent is selected from gelatin, pectin, agar, alginate and carrageenan, and mixtures thereof.

11. (canceled)

12. (canceled)

13. A tanning composition according to claim 1 wherein chewable base comprises a sugar but no sugar alcohol and the sugar is a mixture of glucose and sucrose.

14. A tanning composition according to claim 1 wherein the sugar constitutes from about 60% (w/w) to about 95% (w/w) of the total weight of the pastille composition.

15. (canceled)

16. A tanning composition according to claim 6 which additionally comprises one or more substances that promote natural tanning by facilitating the production of melanin.

17. A tanning composition according to claim 16 wherein the one or more further components are selected from vitamin B2 (riboflavin), vitamin B6, vitamin C and vitamin E.

18. (canceled)

19. A tanning composition according to claim 16 wherein the one or more further components are selected from copper salts, L-tyrosine and 4 aminobenzoic acid (PABA).

20. A tanning composition according to claim 6 wherein the skin colour-promoting agents comprise:
(a) β-carotene in an amount of 0.1%-0.8% (w/w);
(b) lutein in an amount of 0%-0.15% (w/w);
(c) lycopene in an amount of 0%-0.1% (w/w);
(d) astaxanthin in an amount of 0%-0.1% (w/w);
(e) zeanxhantin in an amount of 0%-0.1% (w/w); and
(f) a physiologically acceptable copper salt in an amount of 0%-0.05% (w/w);
provided that the total amount of skin colour-promoting agent is the range from 0.5%-1% (w/w).

21. A tanning composition according to claim 1 having the following composition:

| | % (w/w) of total weight |
|---|---|
| Component | of compositions |
| Sucrose | 35-55 |
| Glucose | 30-50 |
| Water | 5-10 |
| Pectin | 2-5 |
| Flavouring agent | 0-2 |
| Citric acid | 1-3 |
| Colouring agent | 0-0.2 |
| Vegetable (e.g. | 0-0.01 |
| sunflower) oil | |
| Natural wax (e.g. | 0-0.01 |
| Carnauba wax) | |
| Tyrosine | 1-4 |
| Vitamin C | 0.5-4 |
| Vitamin A | 0.2-1 |
| (β-carotene) | |
| Vitamin E | 0.05-0.5 |
| Lutein | 0.05-0.2 |
| Zinc | 0-0.1 |
| Lycopene | 0.02-0.1 |
| Vitamin B2 | 0-0.06 |
| (Riboflavin) | |
| Asthaxin | 0.01-0.05 |
| Copper citrate | 0.005-0.03 |
| Zeanxhantin | 0.005-0.02 |
| Selenium | 0-0.003 |
| Total | 100% |

22. A tanning composition having the following composition:

| | % (w/w) of total weight |
|---|---|
| Component | of compositions |
| Sucrose | 35-55 |
| Glucose | 30-50 |
| Water | 5-10 |
| Tyrosine | 1-5 |
| Pectin | 2-5 |
| PABA | 1-5 |
| Flavouring agent | 0-2 |
| Citric acid | 1-3 |
| Vitamin C | 0.2-2 |
| Vitamin E | 0.05-0.5 |
| Zinc citrate | 0-0.1 |
| Vitamin B2 | 0-0.1 |
| (Riboflavin) | |
| Vitamin B12 | 0-0.05 |
| Copper citrate | 0.005-0.030 |
| Vitamin D | 0-0.005 |
| Vegetable (e.g. | 0-0.01 |
| sunflower) oil | |
| Natural wax (e.g. | 0-0.01 |
| Carnauba wax) | |
| Total | 100% |

23. A tanning composition for oral administration by humans, the composition being a gummy candy comprising:
(i) a chewable gummy base comprising a gelling agent and a sugar and/or sugar alcohol; and
(ii) one or more skin colour-promoting agents;
wherein the gelling agent, sugar and/or sugar alcohol and one or more skin colour-promoting agents are as defined in claim 1.

24. A method of cosmetic tanning or cosmetic skin colour enhancement, which method comprises the oral administration to a subject of an effective number of a tanning composition as defined in claim 1.
